(19) United States
(12) Patent Application Publication
(10) Pub. No.: US 2017/0143864 A1
(43) Pub. Date: May 25, 2017

(54) SMART DIFFUSER

(71) Applicant: UNIST (ULSAN NATIONAL INSITUTE OF SCIENCE AND TECHNOLOGY), Ulsan (KR)

(72) Inventors: Jin-Sung Kim, Ulsan (KR); Cha-Joong Kim, Ulsan (KR); Kwan-Myung Kim, Ulsan (KR); Young-Woo Park, Ulsan (KR); Ji-Hyun Lim, Ulsan (KR); Yoon-ki Ahn, Ulsan (KR)

(73) Assignee: UNIST (ULSAN NATIONAL INSITUTE OF SCIENCE AND TECHNOLOGY), Ulsan (KR)

(21) Appl. No.: 15/354,310

(22) Filed: Nov. 17, 2016

(30) Foreign Application Priority Data
Nov. 20, 2015 (KR) 10-2015-0163485

(51) Int. Cl. A61L 9/12; B05B 12/12

(52) U.S. Cl. CPC A61L 9/12; B05B 12/12; A61L 2209/111; A61L 2209/133

(57) ABSTRACT

The present invention relates to a smart diffuser, such as a smart diffuser for sensing environmental information on the inside of a storage space such as a closet, emitting a diffusing agent, and transmitting the environmental information by means of near field communication, thereby continuously monitoring and managing the inside of the storage space.

### CROSS-REFERENCE TO RELATED APPLICATIONS

This application claims priority from Korean Patent Application No. 10-2015-0163485, filed on Nov. 20, 2015, in the Korean Intellectual Property Office. The entire contents of said application are incorporated herein by reference.

### BACKGROUND OF THE DISCLOSURE

Technical Field

The present invention relates to a smart diffuser, more specifically to a smart diffuser for sensing environmental information on the inside of a storage space such as a closet, emitting a diffusing agent, and transmitting the environmental information by means of near field communication, thereby continuously monitoring and managing the inside of the storage space.

Background art

Storage spaces such as closets or dress rooms store a plurality of clothes and daily supplies, so they are very sensitive to temperature, humidity, etc. This is because the condition of clothes may be changed to an extent that they cannot be worn depending on environmental information such as temperature, humidity, etc. Accordingly, it is necessary to control the temperature, humidity, etc. to manage clothes, etc. stored in the closets or dress rooms. In general, closets or dress rooms are open and closed only for using or storing the clothes or daily supplies. Thus, the temperature and humidity are rarely managed, but instead, dehumidifying agents are simply hung on hangers in the closets or dress rooms.

In addition, bad smell may occur by clothes, etc. which have been stored for a long time in the closets or dress rooms, and thus, for example, a diffusing agent injector installed in bathrooms, etc. which periodically emits a diffusing agent in order to remove smell may be installed in the inside of the closets or dress rooms. However, this diffusing agent injector has a battery and a diffusing agent cartridge formed separately. Thus, in case where the battery is still left even when the diffusing agent is used up, emission would be made without the diffusing agent, so there is a problem that this situation cannot be known well.

Prior Arts, Patent Documents

Korean Patent Laid-Open No. 10-2012-0107174

### SUMMARY OF INVENTION

Technical Task

The present invention was derived in order to improve all of the above problems. The object of embodiments of the present invention is to provide a smart diffuser installed in a storage space such as a closet, etc., including a sensing main body and a receiver, wherein the sensor main body includes a diffusing agent cartridge integrally formed with a battery, a sensor and a wireless module, which emits a diffusing agent by the diffusing agent cartridge as the sensor senses environmental information such as temperature, humidity, bad smell, etc. of the storage space, and transmits the environmental information to the receiver by means of near field communication and monitors the information, thereby continuously managing the storage space.

In order to achieve the above-mentioned object, the smart diffuser installed in a storage space according to embodiments of the present invention includes a sensing main body including a diffusing agent cartridge integrally formed with a battery, a sensor sensing environmental information on the inside of the storage space to allow a diffusing agent to be emitted from the diffusing agent cartridge, and a wireless module allowing the environmental information sensed by the sensor to be transmitted by means of near field communication, and a receiver receiving the environmental information sensed by the sensing main body and monitoring the information.

It is preferable that when the diffusing agent of the diffusing agent cartridge is used up, the integrally formed battery is to be replaced together with the diffusing agent.

The environmental information on the inside of the storage space is temperature, humidity and bad smell.

It is preferable that the wireless module is Bluno, a Bluetooth embedded Arduino.

It is preferable that the receiver is a smart phone or a tablet PC of a terminal-type, or a personal computer (PC).

The storage space is a closet, a dress room or a trunk of a vehicle.

It is preferable that the sensing main body further includes a buzzer ringing an alarm according to the environmental information on the inside of the storage space sensed from the sensor.

It is preferable that the sensing main body further includes an LED displaying the environmental information on the inside of the storage space sensed from the sensor or full charge or discharge of the battery.

The smart diffuser of embodiments of the present invention induces the user to remove bad smell by monitoring the smell in the closet through a sensor. Also, once the bad smell is sensed, the diffusing agent cartridge integrally formed with the battery is configured to inject the diffusing agent instantaneously.

Additionally, since the diffusing agent cartridge is integrally formed with the battery, even when the diffusing agent is used up, the battery is replaced at the same time. Accordingly, the bad smell, etc. of the storage space such as the closet can be managed continuously.

Also, a plurality of sensing main bodies may be managed with one terminal including the receiver. Accordingly, the condition of the storage space such as a plurality of closets can be monitored continuously.

### BRIEF DESCRIPTION OF THE DRAWINGS

FIG. 1 is a perspective view illustrating an example of a smart diffuser according to an embodiment of the present invention; and

FIG. 2 is an exploded perspective view illustrating the inside of an example of the smart diffuser according to an embodiment of the present invention.

### DETAILED DESCRIPTION FOR CARRYING OUT THE INVENTION

Hereinafter, preferable embodiments of the smart diffuser according to the present invention will be explained in detail with reference to the drawings attached. The present invention is not limited by the embodiments described herein after, but may be implemented in various ways. The present embodiments are simply provided in order to complete the disclosure of the present invention and completely inform a skilled person in the art of the scope of the invention.

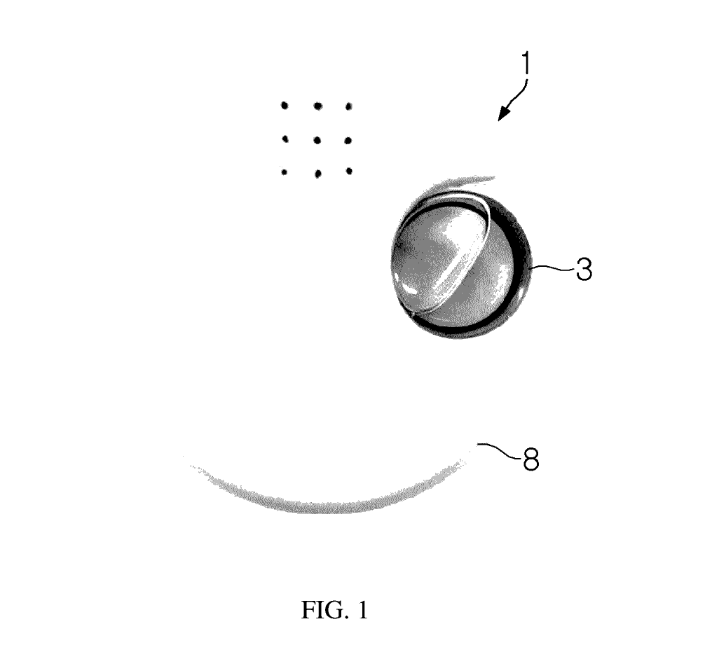

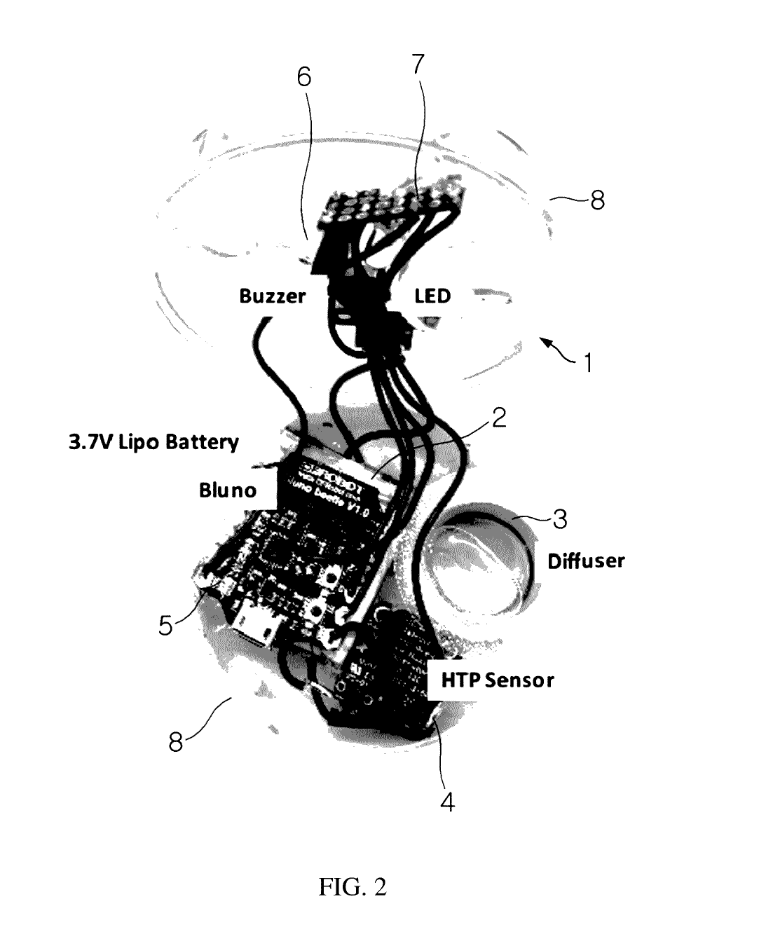

FIG. 1 is a perspective view illustrating an example of a smart diffuser according to embodiments of the present invention, and FIG. 2 is an exploded perspective view illustrating the inside of an example of the smart diffuser according to embodiments of the present invention.

As illustrated in FIGS. 1 and 2, the smart diffuser according to embodiments of the present invention includes a sensing main body 1 and a receiver. The sensing main body 1 includes a diffusing agent cartridge 3 integrally formed with a battery 2, a sensor 4 and a wireless module 5. The sensing main body 1, for example, is formed like a plastic discus-shaped container 8 with a low height as in FIG. 1 and installed in the storage space such as the closet, dress room or trunk of a vehicle. The receiver is used separately from the sensing main body 1.

As an example of the sensing main body 1, the diffusing agent cartridge 3 as a diffuser in which the diffusing agent is injected and emitted to a U-shaped groove formed at one side of the plastic discus-shaped container 8 is exposed and installed. Additionally, the diffusing agent cartridge 3 is integrally installed with a 3.7V Lipo battery 2 in the plastic discus-shaped container 8 forming the sensing main body 1. An HTP sensor including a sensor 4 and a Bluetooth embedded Arduino, Bluno, including a wireless module 5 are electrically connected to each other and installed together. The Bluno, the Bluetooth embedded Arduino, performs near field communication by Bluetooth and plays a role of a controller which controls a sensing drive, etc. of the sensor 4 by Arduino. Additionally, a buzzer 6 and an LED 7 electrically connected with the wireless module 5 are installed and controlled by Arduino of the wireless module 5.

The receiver may include a smart phone or a tablet PC as a terminal interlocking with the sensing main body 1, and a personal computer (PC) may be used as the receiver.

The sensor 4 of the sensing main body 1 senses the temperature, humidity, bad smell, etc. in the closet. In case of sensing bad smell, the sensor emits the diffusing agent from the diffusing agent cartridge 3 by the control of Arduino embedded in the wireless module 5 to remove bad smell in the closet. Additionally, in case of sensing environmental information such as temperature, humidity, etc. by the sensor 4, the information is transmitted by means of near field communication using Bluetooth embedded in the wireless module 5 and received by a receiver like the smart phone, etc., so as to continuously monitor the condition of the inside of the closet. In this case, the diffusing agent cartridge 3 and battery 2 are integrally formed in the sensing main body 1 so the battery 2 is replaced at the same time when the diffusing agent is used up. Thus, the inside of the closet may be continuously managed by sensing the emission of the diffusion agent by sensing bad smell, temperature, humidity, etc., and monitoring them. In addition, when transmitting environmental information on the inside of the closet such as temperature, humidity, etc. to the receiver by means of near field communication, the information is transmitted to a neighboring server. The server transmits the information to a terminal such as a smart phone, etc. of a user used as the receiver, if necessary, in order to perform the monitoring.

Also, in case where not only one sensing main body 1 is installed, but a plurality of sensing main bodies 1 are installed at various places such as closets or other storage spaces, the plurality of sensing main bodies 1 are managed by one terminal such as a smart phone, etc., so that the condition of the plurality of storage spaces can be monitored continuously.

Meanwhile, the buzzer 6 is used to ring an alarm according to the environmental information in the closet sensed by the sensor 4 or to inform a replacement time of the battery. The LED 7 is used to display the environmental information such as the temperature, humidity, etc. in the closet or full charge or discharge of the battery.

According to the smart diffuser of embodiments of the present invention, the sensing main body 1 may be connected to the personal computer (PC) by means of near field communication. Thus, the smart diffuser of the present invention may be used in association with home automation that has already been built and may be used in association with Internet of Things (IoT) in the future.

The smart diffuser according to embodiments of the present invention was explained with reference to the drawings attached. However, the present invention is not limited by the embodiments and drawings described in the present specification, but various modifications may be made by a skilled person in the art within the scope of technical knowledge of the present invention.

### DESCRIPTION ON REFERENCE NUMERALS

1: Sensing main body
2: Battery
3: Diffusing agent cartridge
4: Sensor
5: Wireless module
6: Buzzer
7: LED
8: Plastic discus-shaped container

What is claimed is:

1. A smart diffuser installed in a storage space, comprising:
a sensing main body comprising a diffusing agent cartridge integrally formed with a battery, a sensor sensing environmental information on the inside of the storage space to allow a diffusing agent to be emitted from the diffusing agent cartridge, and a wireless module allowing the environmental information sensed by the sensor to be transmitted by means of near field communication; and
a receiver receiving the environmental information sensed by the sensing main body and monitoring the information.

2. The smart diffuser of claim 1, wherein when the diffusing agent of the diffusing agent cartridge is used up, the integrally formed battery is to be replaced together with the diffusing agent.

3. The smart diffuser of claim 1, wherein the environmental information on the inside of the storage space is temperature, humidity, and bad smell.

4. The smart diffuser of claim 1, wherein the wireless module is Bluno, a Bluetooth embedded Arduino.

5. The smart diffuser of claim 1, wherein the receiver is a smart phone or a tablet PC of a terminal-type, or a personal computer (PC).

6. The smart diffuser of claim 1, where in the storage space is a closet, a dress room, or a trunk of a vehicle.

7. The smart diffuser of claim 1, wherein the sensing main body further comprises a buzzer ringing an alarm according to the environmental information on the inside of the storage space sensed from the sensor.

8. The smart diffuser of claim 1, wherein the sensing main body further comprises an LED displaying the environmental information on the inside of the storage space sensed from the sensor of full charge or discharge of the battery.
